(19) United States
(12) Patent Application Publication
(10) Pub. No.: US 2025/0244241 A1
(43) Pub. Date: Jul. 31, 2025

(54) OPTICAL NANOSENSORS FOR HYDROLYTIC ENZYME ACTIVITY ON SOLID SUBSTRATES

(71) Applicant: Iowa State University Research Foundation, Inc., Ames, IA (US)

(72) Inventors: Nigel Forest Reuel, Ames, IA (US); Nathaniel Kallmyer, Whitestown, IN (US)

(21) Appl. No.: 18/854,023

(22) PCT Filed: Apr. 4, 2023
(86) PCT No.: PCT/US2023/017473

§ 371 Date: Oct. 3, 2024

Related U.S. Application Data
(60) Provisional application No. 63/328,116, filed on Apr. 6, 2022.

(51) Int. Cl. G01N 21/64; G01N 21/77

(52) U.S. Cl. CPC G01N 21/6428; G01N 21/77; G01N 2021/6432; G01N 2021/6439; G01N 2021/7786

(57) ABSTRACT

Various aspects of the instant disclosure relate to a sensor assembly probe for determining enzymatic activity. The sensor assembly probe includes a fluorescent semi-conductive nanoparticle and a solid phase substrate for a predetermined enzyme, the fluorescent semi-conductive nanoparticle adsorbed thereto or at least partially embedded therein.

### CROSS-REFERENCE TO RELATED APPLICATION

This application claims the benefit of priority to U.S. Provisional Patent Application Ser. No. 63/328,116 entitled “OPTICAL NANOSENSORS FOR HYDROLYTIC ENZYME ACTIVITY ON SOLID SUBSTRATES,” filed Apr. 6, 2022, the disclosure of which is incorporated herein in its entirety by reference.

### STATEMENT OF GOVERNMENT SUPPORT

This invention was made with Government support under Award No. 2019-67011-29517 awarded by USDA/NIFA, and under Contract No. FA8650-15-D-5405 awarded by the United States Department of Defense. The U.S. Government has certain rights in this invention.

### BACKGROUND

Determining the presence and activity of an enzyme can be useful in many different contexts. In some applications, this can be done indirectly by detecting the presence of byproducts of the reaction between an enzyme and substrate. Indirect detection can be unreliable and potentially expensive. It may, therefore, be desirable to develop improved detection methods and assemblies.

### SUMMARY OF THE DISCLOSURE

Various aspects of the instant disclosure relate to a sensor assembly probe for determining enzymatic activity. The sensor assembly probe includes a fluorescent semi-conductive nanoparticle and a solid phase substrate for a predetermined enzyme, the fluorescent semi-conductive nanoparticle adsorbed or at least partially embedded thereto.

There are many advantages associated with the assemblies and methods disclosed herein, some of which are unexpected. For example, according to various embodiments, the assembly can be easily and rapidly synthesized and detect the presence of an enzyme and depletion of an enzyme substrate or analogue thereof in real time. Additionally, according to various embodiments, even though the substrate can be fluorogenic or colorimetric, it is not necessary and therefore a natural substrate analogue can be used. According to various embodiments, the assembly is versatile, for example, it can screen different types of substrate and enzyme combinations. According to various embodiments, depletion of substrate can be correlated with signal change to predict quantitative rate constants. According to various further embodiments, comparison with an established colorimetric assay can demonstrate high performance in complex, otherwise-difficult samples. According to various further embodiments, the assemblies and methods disclosed herein can be used to rapidly track changes in enzyme activity to monitor damage or to perform optimization operations.

According to further embodiments, the assembly's construction allow for testing the degradation of a solid-phase substrate. Accordingly an analogue of a solid-phase substrate does not have to be used to confirm whether an enzyme for a predetermined solid-phase substrate is present. According to further embodiments, the probes and methods described herein can be used to evaluate the usefulness of biocatalysts used to degrade recalcitrant synthetic polymers.

### BRIEF DESCRIPTION OF THE FIGURES

The drawings illustrate generally, by way of example, but not by way of limitation, various embodiments discussed in the present document.

FIGS. 1A, 1B and 1C are a schematic views showing a method of making a sensor assembly.

FIG. 2A shows responses of paper/SWNT system to samples with addition occurring at approximately 300 seconds. Shaded regions indicate a single standard deviation (n=4).

FIG. 2B shows responses of PLA-paper/SWNT immediately after sample addition. Shaded regions indicate a single standard deviation (n=4).

### DETAILED DESCRIPTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “weight-average molecular weight” as used herein refers to Mw, which is equal to ΣMᵢ²nᵢ/ΣMᵢnᵢ, where nᵢ is the number of molecules of molecular weight Mᵢ. In various examples, the weight-average molecular weight can be determined using light scattering, small angle neutron scattering, X-ray scattering, and sedimentation velocity.

This disclosure is directed towards various embodiments of a sensor assembly probe. The sensor assembly probe can be used for determining enzymatic activity. Specifically, the sensor assembly probe can be used to determine enzymatic activity for an enzyme used to degrade a solid-state substrate. The sensor assembly can include one or more fluorescent semi-conductive nanoparticles adsorbed or at least partially embedded within or fully embedded within a solid-state substrate.

In operation, the nanoparticles have a detectible fluorescent emission when adsorbed to the solid-state substrate. The nanoparticles are coated with a hydrophobic surfactant. The hydrophobic surfactant helps the nanoparticle to adsorb on the solid-state substrate.

According to one non-limiting theory, upon contact with the predetermined enzyme, the solid-state substrate is degraded. Degradation can occur by bonds in the solid-state substrate being hydrolyzed or otherwise cleaved, for example, by removing a charged group. This results in the nanoparticles being released and adhering to each other. This quenches or at least decreases the fluorescent signal of the nanoparticles. The change in fluorescent signal indicates that the predetermined enzyme of interest is present in solution. According to another non-limiting theory, the release degradation can result in the release of the nanoparticles which can limit access to the solvent present and a quenched signal.

In some examples, instead of signal quenching or reduction being indicative of degradation, an increase in signal intensity can be indicative. For example, it is possible for the solution to which the nanoparticles are disposed in can include brightening agents such as a surfactants of small organic molecules capable of adhering to the surface of the nanoparticles both of which can help to prevent or mitigate aggregation of the nanoparticles such that the fluorescent signal is not quenched.

The nanoparticles can include any suitable material. Examples of suitable materials can include a ceramic material (e.g., aluminum oxide or copper (II) oxide), a polymer, a glass-ceramic, a composite, a metal carbide (e.g., SiC), a nitride (e.g., aluminum nitride, silicon nitride), a metal (e.g., Al, Cu, Au, Ag), a non-metal (e.g., graphite and carbon). The nanoparticle can have any suitable morphology. For example, the morphology of the nanoparticle can be chosen from a nanosphere, a nanorod, a nanofiber, a nanotube, a nanostar, a nanocup, or combinations thereof. At least one of a length, width, and diameter of the nanoparticle is in a range of from about 0.5 nm to about 10,000 nm, about 1 nm to about 100 nm, about 10 nm to about 50 nm, about 100 nm to about 2,500 nm, about 2,500 nm to about 10,000 nm, or less than, equal to, or greater than about 0.5 nm, 0.7, 1, 25, 50, 100, 500, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, or about 10,000 nm. Generally, nanoparticles in which at least one of a length, width, and diameter of the nanoparticle is in a range of from about 0.5 nm to about 100 nm are classified as ultrafine nanoparticles. Generally, nanoparticles in which at least one of a length, width, and diameter of the nanoparticle is in a range of from about 100 nm to about 2,500 nm are classified as fine nanoparticles. Generally, nanoparticles in which at least one of a length, width, and diameter of the nanoparticle is in a range of from about 2,500 nm to about 10,000 nm are classified as coarse nanoparticles. The morphology of the nanoparticles can be uniform.

The sensor assembly probe can include a plurality of the nanoparticles. Respective individual nanoparticles can have at least one of substantially the same morphology, substantially the same dimensions, and have substantially the same composition. Alternatively, the respective individual nanoparticles can differ in at least one of their morphologies, dimensions, and compositions. The plurality of nanoparticles can be heterogeneously or homogenously distributed in the aqueous medium.

When at least one nanoparticle is adsorbed, partially embedded, or fully embedded in the solid-state substrate, the nanoparticle fluoresces. In some embodiments, the nanoparticle can fluoresce at wavelengths ranging from about 800 nm to about 1500 nm, 950 nm to about 1100 nm, or less than, equal to, or greater than about 800, 810, 820, 830, 840, 850, 860, 870, 880, 890, 900, 910, 920, 930, 940, 950, 960, 970, 980, 990, 1000, 1100, 1200, 1300, 1400, or about 1500 nm. In embodiments where the sensor assembly probe includes a plurality of nanoparticles, respective nanoparticles can fluoresce at substantially the same frequency.

In other embodiments where the sensor assembly includes a plurality of nanoparticles, respective nanoparticles can fluoresce at different frequencies. For example, the respective fluorescent signals emitted by the first fluorescent semi-conductive nanoparticle and the second fluorescent semi-conductive nanoparticle have intensities of fluorescence that differ by about 0 to 100%, about 0 to 20% or less than, equal to, or greater than about 0%, 5, 10, 15, 20, 25, 30, 35, 40, 45, 50, 55, 60, 65, 70, 75, 80, 85, 90, 95, or 100%.

The solid-state substrate for the predetermined enzyme can be selected from many suitable candidates. In some examples, the solid-state substrate can include a bond that is hydrolyzable by the predetermined enzyme. Examples of such bonds can include an ester bond, a glycosylic bond, an ether bond, a peptide bond, an acid anhydride bond, a halide bond, a phosphorous-sulfur bond, a sulfur-sulfur bond, a carbon-phosphorous bond, a carbon-sulphur bond, or a combination thereof. The solid-state substrate can include paper, plastic, metal, a ceramic, or a combination thereof.

The predetermined enzyme in the sensor probe assembly can be any suitable enzyme. The enzyme is selected to react with the substrate analogue. The sensor assembly probe can include more than one types of enzyme. In embodiments that include more than one enzyme, the enzymes can be the same enzyme or a mixture of different enzymes. Where different enzymes are present in the assembly, the different enzymes can be adapted to react with different substrate analogues. The substrate analogue that a particular enzyme reacts with may be present in the assembly or may not be present in the assembly.

The predetermined enzyme or enzymes may belong to any class of enzymes. For example, the enzyme or enzymes may be classified as a hydrolase (alternatively known as an EC 3 enzyme). The hydrolase can be classified by the bond it acts upon. For example, the hydrolase can be chosen from a phytase, an esterase, nuclease, phosphodiesterase, lipase, phosphatase, DNA glycosylase, glycoside hydrolase, proteases, peptidase, acid anhydride hydrolase, helicase, GTPase, or mixtures thereof. A cellulase can also be the predetermined enzyme. Examples of a protease can include a cysteineprotease, a serineprotease, a threonineprotease, an aspartic protease, a glutamic protease, a metalloprotease, a PA clan protease, or a mixture thereof. Examples of a cellulose can include endo-1,4-beta-D-glucanase (beta-1,4-glucanase, beta-1,4-endoglucan hydrolase, endoglucanase D, 1,4-(1,3,1,4)-beta-D-glucan 4-glucanohydrolase), carboxymethyl cellulase (CMCase), avicelase, celludextrinase, cellulase A, cellulosin AP, alkali cellulase, cellulase A 3, 9.5 cellulase, and pancellase SS. Examples of oxidases can include glucose oxidase, monoamine oxidase, cytochrome p450 oxidase, NADPH oxidase, xanthine oxidase, L-gulonolactone oxidase, laccase, lysyl oxidase, polyphenol oxidase, sulfhydryl oxidase, or a mixture thereof.

The sensor assembly further includes a porous support substrate. The solid phase substrate is disposed on the porous support substrate. The porous support substrate serves to provide a platform for the solid phase substrate. The porosity of the porous support substrate can allow for the solid phase substrate to at least partially penetrate the porous support substrate. The porous support substrate can include paper, plastic, a composite, a protein, a polysaccharide, an oligosaccharide, or a mixture thereof.

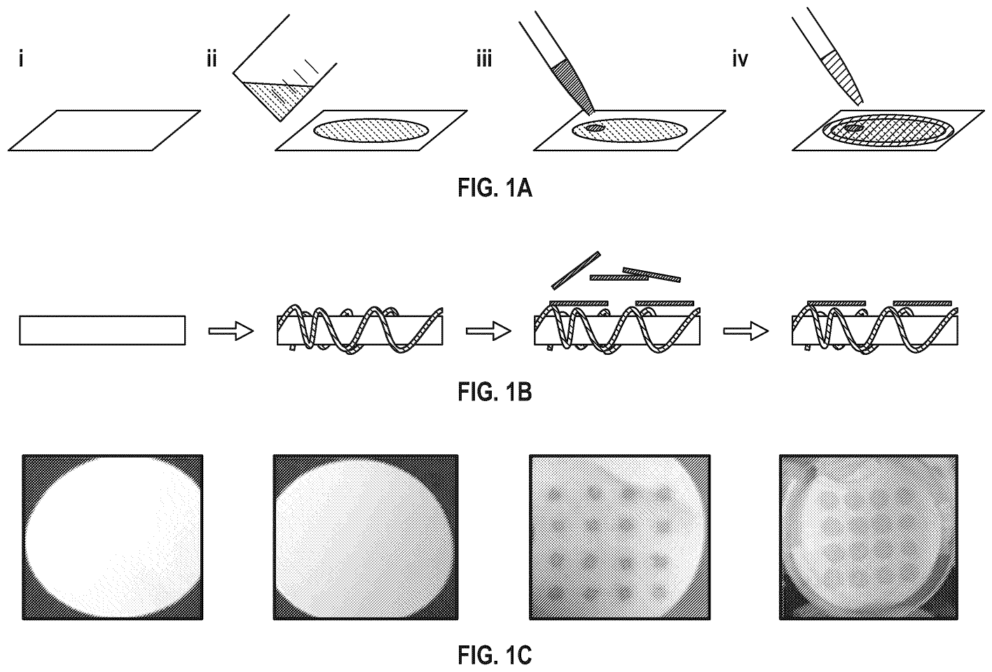

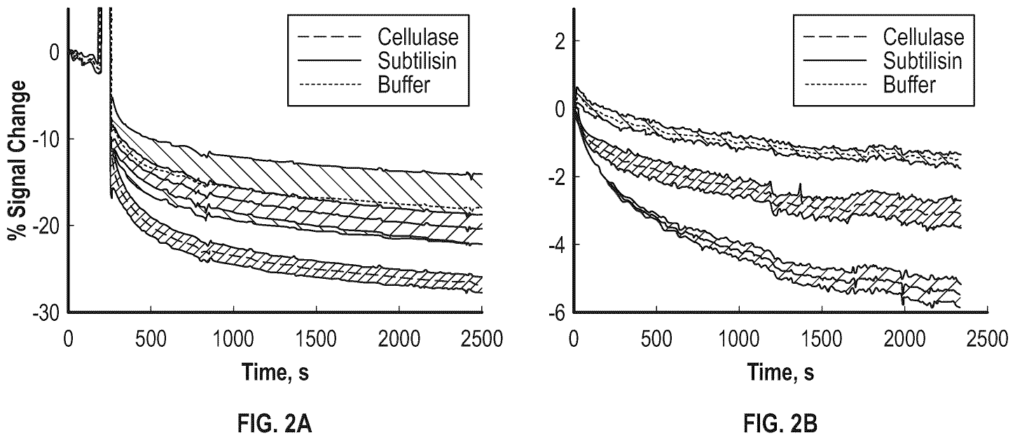

The sensor assembly can be used according to any suitable method. According to various embodiments, the method can include dispensing the nanoparticles to the solid-state substrate so that it is adsorbed thereto or partially embedded or fully embedded within. When the nanoparticles are adsorbed, they produce a fluorescent emission. The initial fluorescence is measured. In some embodiments where the assembly includes a mixture of nanoparticles that produce different fluorescent emissions, multiple emissions may be measured. FIG. 1 is a schematic picture showing a method of making the sensor assembly. As shown a porous substrate (the white square and gray tube) is contacted with the solid phase substrate for the predetermined enzyme (solid phase substrate shown as a green material). Subsequently, one or more fluorescent semi-conductive nanoparticles are contacted with the solid phase substrate (fluorescent semi-conductive nanoparticles shown as black tubes). Finally a solution (the blue liquid) including the predetermined enzyme is dispensed on the assembly.

The enzyme or mixture of enzymes are then contacted with the nanoparticles and solid-state substrate analogues. If an enzyme is associated with a particular solid-state substrate, the solid-state substrate will be degraded and release the nanoparticles. When the nanoparticles are released, their fluorescence can decrease or disappear. The fluorescent signal then disappears as a result of the nanoparticles agglomerating and their signal being quenched. Thus, a measured second fluorescent emission will have a different intensity than the first fluorescent emission or there will be no fluorescent emission and will be indicative of the substrate being degraded.

Measuring a second fluorescent emission that is different than the first fluorescent emission confirms the presence of a predetermined enzyme. In embodiments where nanoparticles having different fluorescent emissions and different substrates attached thereto are present, a decrease in the emission in one or both of the nanoparticles can indicate the presence of two different predetermined enzymes. In this manner, the presence of one or more enzymes in a mixture of enzymes or another constituent of a solution can be confirmed. Additionally, the rate of reaction between the predetermined enzyme and a substrate analogue can be determined by monitoring the rate at which the fluorescent emission intensity changes.

### Examples

Various embodiments of the present disclosure can be better understood by reference to the following Examples which are offered by way of illustration. The present disclosure is not limited to the Examples given herein.

### Preparation of Sensors

Cholate-single wall nanotube (SWNT) were prepared by first dissolving cholate (Sigma C6445) in deionized water to a concentration of 2.5 mg/mL. SWNT were then added to 2 mL of this solution at a ratio of 0.5 mg per mL solution. This mixture was then sonicated by probe tip sonication (Qsonica Q125, ⅛″) at 2 W for 1 h.

Filter paper was placed in a vacuum for 30 min. For cellulase sensors, 3 μL cholate-SWNT were spotted onto filter paper (Whatman filter paper grade 4) with the pattern of a 96-well plate. This paper was allowed to sit for 5 min to allow SWNT to adsorb. Lastly, this paper was rinsed with deionized water to remove unbound SWNT.

For coatings, polylactic acid (PLA) from 3D printer resin was dissolved until saturated in tetrahydrofuran for two days. Next, vacuum treated, unspotted filter paper was submerged in the PLA solution and allowed to dry. This was repeated once. SWNT were then spotted onto the PLA-paper by the procedure above.

### Degradation Assay

Spotted filter papers were tested with a custom fluorescent plate reader. Prior to scanning, filter papers were made damp with pH 5 McIlvane buffer. Papers were mounted in a petri dish taped to sample stage of the plate reader. To prevent the paper from drying, a wet paper towel was placed in the top half of the Petri dish. Adhesion to the top of the petri dish was sufficient to hold the towel in place. Scans were run for approximately 20 min. 2 minutes into the scan, 3 μL samples were added to each spot. Samples included different concentrations of cellulase (Viscozyme®, Sigma V2010) and subtilisin (Sigma 8460). Both enzymes were received as suspensions, so concentrations were reported as percentages of the original. Enzyme solutions were diluted in pH 5 buffer. Denatured enzyme was prepared by sonicating 50% cellulase solution at 10 W for 15 min. Data was plotted with Matlab.

### Results and Discussion

Sensors were tested with cellulase, subtilisin, and a buffer control (FIG. 3). While these tests did show relatively high background effects, target enzymes produced the largest responses by significant margins. Paper/SWNT were most sensitive to cellulase, and PLA-paper/SWNT were most sensitive to subtilisin. Cellulase did produce a significant response from the PLA-paper/SWNT system; however, this was an expected residual effect of the paper support, as any exposed cellulose would remain susceptible to cellulolytic degradation. Thus, optimization of the paper-coating process may reduce this residual sensitivity. More importantly, these tests demonstrated that selectivity can be easily altered by adding different coatings to the paper. The background observed with buffer controls is suspected to be an effect of drying, which would gradually increase salt concentration and quench SWNT fluorescence12. Although sensors were kept in a petri dish with a damp paper towel, this indicates a need for more effective and consistent humidity control.

### Additional Embodiments

The following exemplary embodiments are provided, the numbering of which is not to be construed as designating levels of importance:

Aspect 1 provides a sensor assembly probe for determining enzymatic activity, the sensor assembly probe comprising:
a fluorescent semi-conductive nanoparticle;
a solid phase substrate for a predetermined enzyme, the fluorescent semi-conductive nanoparticle adsorbed or at least partially embedded thereto; and
a porous support substrate having the solid phase substrate disposed thereon.

Aspect 2 provides the sensor assembly probe of Aspect 1, wherein a morphology of the fluorescent semi-conductive nanoparticle comprises a nanosphere, a nanorod, a nanofiber, a nanotube, a nanostar, a nanocup, or combinations thereof.

Aspect 3 provides the sensor assembly probe of any one of Aspects 1 or 2, wherein at least one of a length, width, and diameter of the fluorescent semi-conductive nanoparticle is in a range of from about 0.5 nm to about 100 nm.

Aspect 4 provides the sensor assembly probe of any one of Aspects 1-3, wherein a particle size of the fluorescent semi-conductive nanoparticle is in a range of from about 10 nm to about 50 nm.

Aspect 5 provides the sensor assembly probe of any one of Aspects 1-4, wherein the fluorescent semi-conductive nanoparticle comprises a ceramic, a polymer, a metal carbide, a nitride, a metal, graphite, carbon, or a mixture thereof.

Aspect 6 provides the sensor assembly probe of any one of Aspects 1-5, wherein the fluorescent semi-conductive nanoparticle is a carbon nanotube.

Aspect 7 provides the sensor assembly probe of any one of Aspects 1-6, wherein the fluorescent semi-conductive nanoparticle fluoresces at frequency ranging from about 800 nm to about 1500 nm.

Aspect 8 provides the sensor assembly probe of any one of Aspects 1-7, wherein the fluorescent semi-conductive nanoparticle fluoresces at frequency ranging from about 950 nm to about 1100 nm.

Aspect 9 provides the sensor assembly probe of any one of Aspects 1-8, wherein the fluorescent semi-conductive nanoparticle is at least partially coated with a surfactant.

Aspect 10 provides the sensor assembly probe of Aspect 9, wherein the surfactant is hydrophobic or includes a hydrophobic portion.

Aspect 11 provides the sensor assembly probe of any one of Aspects 9 or 10, wherein the surfactant comprises an anionic surfactant, a cationic surfactant, a zwitterionic surfactant, a non-ionic surfactant, or a mixture thereof.

Aspect 12 provides the sensor assembly probe of any one of Aspects 9-11, wherein the surfactant comprises cholate.

Aspect 13 provides the sensor assembly probe of any one of Aspects 1-12, wherein the solid-phase substrate comprises paper, plastic, a composite, or a mixture thereof.

Aspect 14 provides the sensor assembly probe of any one of Aspects 1-13, wherein the solid-phase substrate comprises a bond that is an ester bond, a urethane bond, a glycosylic bond, an ether bond, a peptide bond, an acid anhydride bond, a halide bond, a phosphorous-sulfur bond, a sulfur-sulfur bond, a carbon-phosphorous bond, a carbon-sulfur bond, or a combination thereof.

Aspect 15 provides the sensor assembly probe of any one of Aspects 1-14, wherein the predetermined enzyme comprises a hydrolase, an oxidase, a cellulase, a protease or a mixture thereof.

Aspect 16 provides the sensor assembly probe of Aspect 15, wherein the hydrolase is chosen from an esterase, a nuclease, a phosphodiesterase, a lipase, a phosphatase, a DNA glycosylase, a glycoside hydrolase, a protease, a peptidase, an acid anhydride hydrolase, a helicase, a GTPase, or a mixture thereof.

Aspect 17 provides the sensor assembly probe of any one of Aspects 15 or 16, wherein the protease comprises a cysteineprotease, a serineprotease, a threonineprotease, an aspartic protease, a glutamic protease, a metalloprotease, a PA clan protease, or a mixture thereof.

Aspect 18 provides the sensor assembly probe of any one of Aspects 15-17, wherein the cellulase comprises endo-1,4-beta-D-glucanase (beta-1,4-glucanase, beta-1,4-endoglucan hydrolase, endoglucanase D, 1,4-(1,3,1,4)-beta-D-glucan 4-glucanohydrolase), carboxymethyl cellulase (CMCase), avicelase, celludextrinase, cellulase A, cellulosin AP, alkali cellulase, cellulase A 3, 9.5 cellulase, and pancellase SS.

Aspect 19 provides the sensor assembly probe of any one of Aspects 15-18, wherein the oxidase comprises glucose oxidase, monoamine oxidase, cytochrome p450 oxidase, NADPH oxidase, xanthine oxidase, L-gulonolactone oxidase, laccase, lysyl oxidase, polyphenol oxidase, sulfhydryl oxidase, or a mixture thereof.

Aspect 20 provides the sensor assembly probe of any one of Aspects 1-19, wherein the fluorescent semi-conductive nanoparticle is a first fluorescent semi-conductive nanoparticle and the assembly further comprises a second fluorescent semi-conductive nanoparticle.

Aspect 21 provides the sensor assembly probe of Aspect 20, wherein the first fluorescent semi-conductive nanoparticle and the second fluorescent semi-conductive nanoparticle have substantially the same composition.

Aspect 22 provides the sensor assembly probe of Aspect 20, wherein the first fluorescent semi-conductive nanoparticle and the second fluorescent semi-conductive nanoparticle have different compositions.

Aspect 23 provides the sensor assembly probe of any one of Aspects 20-22, wherein the first fluorescent semi-conductive nanoparticle and the second fluorescent semi-conductive nanoparticle fluoresce at different frequencies.

Aspect 24 provides the sensor assembly probe of Aspect 23, wherein the respective fluorescent signals emitted by the first fluorescent semi-conductive nanoparticle and the second fluorescent semi-conductive nanoparticle have frequencies of fluorescence that differ by about 0% to about 100%, relative to each other.

Aspect 25 provides the sensor assembly probe of any one of Aspects 23 or 24, wherein the respective fluorescent signals emitted by the first fluorescent semi-conductive nanoparticle and the second fluorescent semi-conductive nanoparticle have frequencies of fluorescence that differ by about 0% to about 20%, relative to each other.

Aspect 26 provides the sensor assembly probe of any one of Aspects 23-25, wherein the first fluorescent semi-conductive nanoparticle and the second fluorescent semi-conductive nanoparticle are homogenously adsorbed about a surface of the solid-phase substrate or at least partially embedded within the surface.

Aspect 27 provides the sensor assembly probe of any one of Aspects 23-26, wherein the first fluorescent semi-conductive nanoparticle and the second fluorescent semi-conductive nanoparticle are heterogeneously adsorbed about a surface of the solid-phase substrate or at least partially embedded within the surface.

Aspect 28 provides a sensor assembly comprising the probe of any one of Aspects 1-27, the sensor assembly further comprising the predetermined enzyme.

Aspect 29 provides the sensor assembly of Aspect 28, wherein the predetermined enzyme is a first enzyme and the assembly further comprises a second enzyme.

Aspect 30 provides the sensor assembly of any one of claims 1-29, wherein the porous substrate comprises paper, plastic, a composite, a protein, a polysaccharide, an oligosaccharide, or a mixture thereof.

Aspect 31 provides the method of using the sensor assembly probe of any one of Aspects 1-30, the method comprising:
measuring a first fluorescent frequency emission of the probe;
contacting the substrate and the predetermined enzyme; and
measuring a second fluorescent frequency emission of the probe, wherein the second fluorescent frequency emission is less than the first fluorescent frequency emission and indicates that at least a portion the substrate has reacted with the predetermined enzyme.

Aspect 32 provides the method of Aspect 30, wherein the second fluorescent frequency emission is zero.

Aspect 33 provides the method of any one of Aspects 31 or 32, wherein a mixture of enzymes comprises the predetermined enzyme.

Aspect 34 provides the method of any one of Aspects 31-33, further comprising determining a rate of reaction between the substrate and the predetermined enzyme.

Aspect 35 provides the method of Aspect 34, wherein determining a rate of reaction comprises measuring a plurality of fluorescent signals over a predetermined amount of time to quantify the amount of substrate that is consumed by the predetermined enzyme.

Aspect 36 provides a method of making the sensor assembly probe of any one of Aspects 1-35, the method comprising:
contacting the fluorescent semi-conductive nanoparticle and the surfactant; and
contacting the fluorescent semi-conductive nanoparticle and the substrate, to form the sensor assembly.

Aspect 37 provides the method of Aspect 36, wherein contacting the fluorescent semi-conductive nanoparticle and the surfactant comprises sonication.

1. A sensor assembly probe for determining enzymatic activity, the sensor assembly probe comprising:
a fluorescent semi-conductive nanoparticle;
a solid phase substrate for a predetermined enzyme, the fluorescent semi-conductive nanoparticle adsorbed or at least partially embedded thereto; and
a porous support substrate having the solid phase substrate disposed thereon.

2. The sensor assembly probe of claim 1, wherein a morphology of the fluorescent semi-conductive nanoparticle comprises a nanosphere, a nanorod, a nanofiber, a nanotube, a nanostar, a nanocup, or combinations thereof.

3. The sensor assembly probe of claim 1, wherein at least one of a length, width, and diameter of the fluorescent semi-conductive nanoparticle is in a range of from about 0.5 nm to about 100 nm.

4. The sensor assembly probe of claim 1, wherein a particle size of the fluorescent semi-conductive nanoparticle is in a range of from about 10 nm to about 50 nm.

5. The sensor assembly probe of claim 1, wherein the fluorescent semi-conductive nanoparticle comprises a ceramic, a polymer, a metal carbide, a nitride, a metal, graphite, carbon, or a mixture thereof.

6. The sensor assembly probe of claim 1, wherein the fluorescent semi-conductive nanoparticle is a carbon nanotube.

7. The sensor assembly probe of claim 1, wherein the fluorescent semi-conductive nanoparticle fluoresces at frequency ranging from about 800 nm to about 1500 nm.

8. The sensor assembly probe of claim 1, wherein the fluorescent semi-conductive nanoparticle fluoresces at frequency ranging from about 950 nm to about 1100 nm.

9. The sensor assembly probe of claim 1, wherein the fluorescent semi-conductive nanoparticle is at least partially coated with a surfactant.

10. The sensor assembly probe of claim 9, wherein the surfactant is hydrophobic or includes a hydrophobic portion.

11. The sensor assembly probe of claim 9, wherein the surfactant comprises an anionic surfactant, a cationic surfactant, a zwitterionic surfactant, a non-ionic surfactant, or a mixture thereof.

12. The sensor assembly probe of claim 9, wherein the surfactant comprises cholate.

13. The sensor assembly probe of claim 1, wherein the solid-phase substrate comprises paper, plastic, a composite, or a mixture thereof.

14. The sensor assembly probe of claim 1, wherein the solid-phase substrate comprises a bond that is an ester bond, a urethane bond, a glycosylic bond, an ether bond, a peptide bond, an acid anhydride bond, a halide bond, a phosphorous-sulfur bond, a sulfur-sulfur bond, a carbon-phosphorous bond, a carbon-sulfur bond, or a combination thereof.

15. The sensor assembly probe of claim 1, wherein the predetermined enzyme comprises a hydrolase, an oxidase, a cellulase, a protease or a mixture thereof.

16. The sensor assembly probe of claim 15, wherein the hydrolase is chosen from an esterase, a nuclease, a phosphodiesterase, a lipase, a phosphatase, a DNA glycosylase, a glycoside hydrolase, a protease, a peptidase, an acid anhydride hydrolase, a helicase, a GTPase, or a mixture thereof.

17. The sensor assembly probe of claim 15, wherein the protease comprises a cysteineprotease, a serineprotease, a threonineprotease, an aspartic protease, a glutamic protease, a metalloprotease, a PA clan protease, or a mixture thereof.

18. The sensor assembly probe of claim 15, wherein the cellulase comprises endo-1,4-beta-D-glucanase (beta-1,4-glucanase, beta-1,4-endoglucan hydrolase, endoglucanase D, 1,4-(1,3,1,4)-beta-D-glucan 4-glucanohydrolase), carboxymethyl cellulase (CMCase), avicelase, celludextrinase, cellulase A, cellulosin AP, alkali cellulase, cellulase A 3, 9.5 cellulase, and pancellase SS.

19. The sensor assembly probe of claim 15, wherein the oxidase comprises glucose oxidase, monoamine oxidase, cytochrome p450 oxidase, NADPH oxidase, xanthine oxidase, L-gulonolactone oxidase, laccase, lysyl oxidase, polyphenol oxidase, sulfhydryl oxidase, or a mixture thereof.

20. The sensor assembly probe of claim 1, wherein the fluorescent semi-conductive nanoparticle is a first fluorescent semi-conductive nanoparticle and the assembly further comprises a second fluorescent semi-conductive nanoparticle.

21-37. (canceled)
